(19) United States
(12) Patent Application Publication
(10) Pub. No.: US 2006/0198827 A1
(43) Pub. Date: Sep. 7, 2006

(54) Engineering vascularized muscle tissue

(76) Inventors: Shulamit Levenberg, Haifa (IL); Jeroen Rouwkema, Wageningen (NL); Robert S. Langer, Newton, MA (US)

Correspondence Address: CHOATE, HALL & STEWART LLP, TWO INTERNATIONAL PLACE, BOSTON, MA, 02110, US

(21) Appl. No.: 11/347,831

(22) Filed: Feb. 3, 2006

Related U.S. Application Data
(60) Provisional application No. 60/650,427, filed on Feb. 4, 2005.
(60) Provisional application No. 60/691,609, filed on Jun. 17, 2005.

(51) Int. Cl. A61K 35/12; C12N 5/06; C12N 5/08

(52) U.S. Cl. 424/93.7; 435/366; 435/354

(57) ABSTRACT

A tissue engineered construct. The construct includes endothelial cells, muscle cells, and a three-dimensional support matrix on which the endothelial cells and the myoblasts are seeded.

This application claims priority from U.S. Patent Applications Nos. 60/650,427, filed Feb. 4, 2005, and 60/691,609, filed Jun. 17, 2005, the entire contents of both of which are incorporated herein by reference

This work was supported by NIH grants HL60435 and EY05318. The government may have certain rights in this invention.

### FIELD OF THE INVENTION

This invention relates to vascularized tissue engineered constructs and methods of making same.

### BACKGROUND OF THE INVENTION

One of the major obstacles in engineering thick, complex tissues such as muscle is the need to vascularize the tissue in vitro. Vascularization in vitro could maintain cell viability during tissue growth, induce structural organization and promote vascularization upon implantation. Many past approaches to engineering new tissue have relied on the host for vascularization. Although this approach has been useful in many tissues, it has not been as successful in thick, highly vascularized tissues such as the muscle (Saxena, et al., Tissue Eng (1999) 5, 525-532; Neumann, et al., Microvasc Res (2003) 66, 59-67; Bach, et al., Clin Plast Surg (2003) 30, 589-599). Skeletal muscle includes individual muscle fibers arranged in parallel. Each fiber is a long, cylindrical multinucleated cell that is surrounded by a cell membrane and connective tissue. Skeletal muscles have an abundant blood vessel supply with branches of blood vessels following the connective tissue components of the muscle (Wigmore, et al., Int Rev Cytol (2002) 216, 175-232; Buckingham, Curr Opin Genet Dev (2001) 11, 440-448). So far, attempts to engineer skeletal muscle tissue have used cultivation of skeletal myoblasts only, in some cases using growth factor delivery matrices or genetically engineered myoblasts to express vascularization factors (Zisch, et al., Cardiovasc Pathol (2003) 12, 295-310; von Degenfeld, et al., Br J Pharmacol (2003) 140, 620-626; Lu, et al., Circulation (2001) 104, 594-599).

### Definitions

“Biomolecules”: The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

“Biocompatible”: The term “biocompatible”, as used herein is intended to describe materials that do not elicit an undesirable detrimental response in vivo.

“Biodegradable”: As used herein, “biodegradable” polymers are polymers that degrade fully (i.e., down to monomeric species) under physiological or endosomal conditions. In preferred embodiments, the polymers and polymer biodegradation byproducts are biocompatible. Biodegradable polymers are not necessarily hydrolytically degradable and may require enzymatic action to fully degrade.

“Growth Factors”: As used herein, “growth factors” are chemicals that regulate cellular metabolic processes, including but not limited to differentiation, proliferation, synthesis of various cellular products, and other metabolic activities. Growth factors may include several families of chemicals, including but not limited to cytokines, eicosanoids, and differentiation factors.

“Polynucleotide”, “nucleic acid”, or “oligonucleotide”: The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least three nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Polypeptide”, “peptide”, or “protein”: According to the present invention, a “polypeptide”, “peptide”, or “protein” comprises a string of at least three amino acids linked together by peptide bonds. The terms “polypeptide”, “peptide”, and “protein”, may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polysaccharide”, “carbohydrate” or “oligosaccharide”: The terms “polysaccharide”, “carbohydrate”, or “oligosaccharide” refer to a polymer of sugars. The terms “polysaccharide”, “carbohydrate”, and “oligosaccharide”, may be used interchangeably. Typically, a polysaccharide comprises at least three sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

“Small molecule”: As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present invention.

“Bioactive agents”: As used herein, “bioactive agents” is used to refer to compounds or entities that alter, inhibit, activate, or otherwise affect biological or chemical events. For example, bioactive agents may include, but are not limited to, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents. In certain embodiments, the bioactive agent is a drug.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, and the United States Pharmacopeia-25/National Formulary-20, published by the United States Pharmcopeial Convention, Inc., Rockville Md., 2001, all of which are incorporated herein by reference.

“Tissue”: as used herein, the term “tissue” refers to a collection of cells of one or more types combined to perform a specific function, and any extracellular matrix surrounding the cells.

### SUMMARY OF THE INVENTION

In one aspect, the invention is a tissue engineered construct including endothelial cells, muscle cells, and a three-dimensional support matrix on which the endothelial cells and the muscle cells are seeded. The construct may promote the formation of one or more of smooth, skeletal, and cardiac muscle tissue. The endothelial cells may be embryonic stem cell-derived endothelial cells or umbilical vein endothelial cells, for example mammalian embryonic stem cell-derived endothelial cells, human embryonic stem cell-derived endothelial cells, or human or mouse umbilical vein endothelial cells. The endothelial cells may be mammalian aortic endothelial cells. The muscle cells may be mammalian myoblasts, e.g., human or mouse myoblasts, or skeletal muscle cells, smooth muscle cells, or cardiomyocytes. The construct may further include fibroblasts, for example, embryonic fibroblasts, e.g., human or mouse embryonic fibroblasts.

The of claim 1, wherein the three-dimensional support matrix may include a mixture of poly(L-lactic acid) and poly(lactic acid-co-glycolic acid), e.g., a 50:50 mixture of poly(L-lactic acid) and poly(lactic acid-co-glycolic acid). The three-dimensional support matrix may be biodegradable or non-biodegradable. The three-dimensional support matrix may include collagen-GAG, collagen, fibrin, PLA, PGA, PLA-PGA co-polymers, poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of the above, mixtures of the above, or adducts of the above.

The three-dimensional support matrix may further include a coating including an agent that promotes cell adhesion, for example, fibronectin, integrins, or oligonucleotides that promote cell adhesion. The cells may be combined with growth-factor reduced Matrigel. The construct may further include a gel that coats internal and external surfaces of the three-dimensional support matrix, e.g., collagen gel, alginate, agar, growth factor-reduced Matrigel, and MATRIGEL™. The gel may further include one or more of laminin, fibrin, fibronectin, proteoglycans, glycoproteins, glycosaminoglycans, chemotactic agents, or growth factors. The construct may further include VEGF or another growth factor, e.g., activin-A (ACT), retinoic acid (RA), epidermal growth factor, bone morphogenetic protein, TGF-β, hepatocyte growth factor, platelet-derived growth factor, TGF-α, IGF-I, IGF-II, hematopoietic growth factors, heparin binding growth factor, peptide growth factors, erythropoietin, interleukins, tumor necrosis factors, interferons, colony stimulating factors, acidic and basic fibroblast growth factors, nerve growth factor (NGF), or muscle morphogenic factor.

In another aspect, the invention is a tissue-engineered muscle construct including a three-dimensional support matrix, a plurality of myotubes disposed within the support matrix, and at least one endothelial vessel structure disposed within the support matrix. The endothelial vessel structure may include at least one vessel-like structure having a lumen.

In another aspect, the invention is a tissue-engineered muscle construct including a three-dimensional support matrix, a plurality of cardiac muscle cells disposed within the support matrix, and at least one endothelial vessel structure disposed within the support matrix. The endothelial vessel structure may include at least one vessel-like structure having a lumen.

In another aspect, the invention is a method of producing a tissue engineered construct. The method includes providing a population of endothelial cells, providing a population of muscle cells, seeding the endothelial cells and the muscle cells on a three-dimensional support matrix, and culturing the seeded cell support matrix in a predetermined medium for a predetermined period of time. Seeding may include suspending the muscle cells and the endothelial cells in growth-factor reduced Matrigel and absorbing a predetermined amount of the suspension into the three-dimensional support matrix. The predetermined medium may include one or more of myoblast medium, endothelial cell medium, embryonic fibroblast medium, and cardiac cell medium. Myoblast medium may include DMEM containing 10% fetal bovine serum, 10% calf serum, and 2.5% HEPES buffer. The medium may be further supplemented with VEGF. The method may further include providing a population of fibroblasts, and seeding may include seeding the fibroblasts with the endothelial cells and the myoblasts on the three-dimensional support matrix.

### BRIEF DESCRIPTION OF THE DRAWINGS

The invention is described with reference to the several figures of the drawing, in which,

FIG. 1. A) Photograph of a PLLA/PLGA scaffold prior to cell seeding. Scale bar=1 mm. B) Light micrographs of tissue sections of 3D scaffolds co-cultured with skeletal myoblasts and endothelial cells (HUVEC) and stained for desmin and myogenin after 3 days and 14 days. Scale bar=50 μm. C) Light micrographs of tissue construct sections immunofluorescently stained using anti-CD31 antibodies (red), anti-desmin antibodies (green) and DAPI for nuclear staining (blue), or stained using anti-CD31 antibodies alone (brown) and counterstained with hematoxylin (blue). Co-cultures: endothelial cells (HUVEC or hES-EC when indicated) were co-seeded with skeletal myoblasts on polymer scaffolds and grown for 10 days. Tri-cultures: HUVEC, skeletal myoblasts, and mouse embryonic fibroblasts were grown in myoblast medium or endothelial medium as indicated and stained using anti-CD31 antibodies for 10 days or one month. Cultured cell numbers (myoblast/endothelial/ fibroblast): 0.5/0.7/0.2×10⁶, bottom picture in endothelial medium: 0.5/0.5/0.5×10⁶. Scale bar=50 μm.

FIG. 2. A) Quantitative comparison of vessel formation in co-culture (Co) and tri-culture (Tri) constructs grown with different cell ratios (cell number×10) and medium conditions (myoblast medium and endothelial medium). Endothelial cell ratio (EC %) is calculated as a percentage of the total cell number. Endothelial cell area corresponds to the percentage of area positively stained with CD31 antibody within the tissue section. Lumen area shows the total area of all the lumens in the section as percentages of total section area. Myo=myoblasts, EC=endothelial cells (HUVEC), F=mouse embryonic fibroblasts. * denotes statistical significance (p<0.05) between the indicated pairs. B) Quantitative comparison of vessels in constructs after two and four weeks in vitro. Co-cultures=myroblasts and endothelial cells (0.8 and 0.6×10⁶ cells per scaffold, respectively). Tri-cultures=myoblasts, fibroblasts and endothelial cells (0.6, 0.2 and 0.6×10⁶ cells per scaffold respectively). * denotes statistical significance (p<0.05) compared to controls. C) Light micrographs of constructs seeded with embryonic fibroblasts and endothelial cells, grown for 2 weeks, and immunostained with human-specific anti-CD31 antibodies (brown) (scale bar=50 μm). D) Fluorescence micrographs of constructs seeded with smooth muscle cells and endothelial cells (HUVEC or hES-EC as indicated) and immunofluorescently double-stained using anti-VWF antibodies (green), anti-SMA antibodies (red) and DAPI for nuclear localization. Scale bar=50 μm. E) Quantitative analysis of endothelial positive area and number of endothelial vessels in tri-culture constructs incubated with control medium or medium supplemented with VEGF (50 ng/ml) or PDGF (5 ng/ml) for 2 weeks and immunoassayed using anti-CD31 antibodies. * denotes statistical significance (p<0.05) compared to controls. The results shown are mean values±SD (n=4).

FIG. 3. Photograph and graph quantifying the intensity of bands in a gel showing the results of RT-PCR analysis using primers for mouse VEGF (mVEGF), human PDGF (hPDGH) and GAPDH for constructs seeded with myoblasts (M)(0.8×10⁶), fibroblasts (F)(0.8×10⁶), endothelial cells (E)(0.7×10⁶), a combination of myoblasts and endothelial cells (ME)(0.8/0.7×10), fibroblasts and endothelial cells (FE)(0.8/0.7×10⁶), or a tri-culture of myoblasts, fibroblasts and endothelial cells (MFE)(0.6/0.2/0.7×10⁶). For each gene, mean pixel intensities of each band (obtained in the linear range of the amplification) were measured and normalized to mean pixel intensities of GAPDH band.

FIG. 4. A) Light micrographs of two-week-old engineered muscle constructs implanted either subcutaneously into immuno-deficient mice (S.C), or intramuscularly into rat Quadriceps muscle (Quad) or mouse abdominal muscle (Abdom) (see methods) after 2 weeks in vivo and immunostaining using anti desmin antibodies or myogenin antibodies. (Control: constructs without cells; muscle area=“m”, Implant area=“i”. Scale bar=100 μm.). B) Light micrographs of constructs sectioned and immunostained using human specific anti-CD31 (hCD31) or anti-VWF antibodies after two weeks in vivo. Scale bar=50 μm. C) Quantitative comparison of the number and size of lectin-perfused vessels in muscle implants between tri-culture constructs seeded with myoblasts (M), endothelial cells (HUVEC) (EC), and embryonic fibroblasts (F), and constructs seeded with myoblasts alone or without cells (no cells). (Cell number×10⁶). Standard derivation error bars relate to total number of perfused vessels (n=3). D) Fluorescence micrograph of constructs perfused with fluorescently labeled lectin (red) after two weeks in vivo, frozen and sectioned, and immunofluorecently stained with anti-CD31 (green). E) Photographs of mice taken with a luciferase-based in vivo imaging system (IVIS) (Cell number×10⁶) and graph indicating the signal detected. The results are mean values (±SD) (n=3). * denotes statistical significance (p<0.05) compared to myoblasts alone (M) or myoblasts+fibroblasts (M+F).

FIG. 5. Quantitative comparison of the number of endothelial vessels in muscle implants seeded with HUVEC or hESC-derived endothelial cells between tri-culture constructs seeded with myoblasts (M), embryonic fibroblasts (F), and either HUVEC or hESC-derived endothelial cells (hES-EC). Numbers of cells (×10⁶) seeded are indicated. The results are mean values (±SD) (n=3).

FIG. 6: Light micrographs of a tissue engineering construct co-seeded with rat cardiomyocytes and HUVEC and stained with anti-CD31 antibodies.

FIG. 7: Higher resolution micrograph of the sample in FIG. 6

FIGS. 8 and 9: Fluorescence micrographs of a tissue engineering construct co-seeded with bovine aortic endothelial cells and mouse embryonic fibroblasts and stained to reveal smooth muscle actin (red) and vWF (green).

### DETAILED DESCRIPTION OF CERTAIN PREFERRED EMBODIMENTS

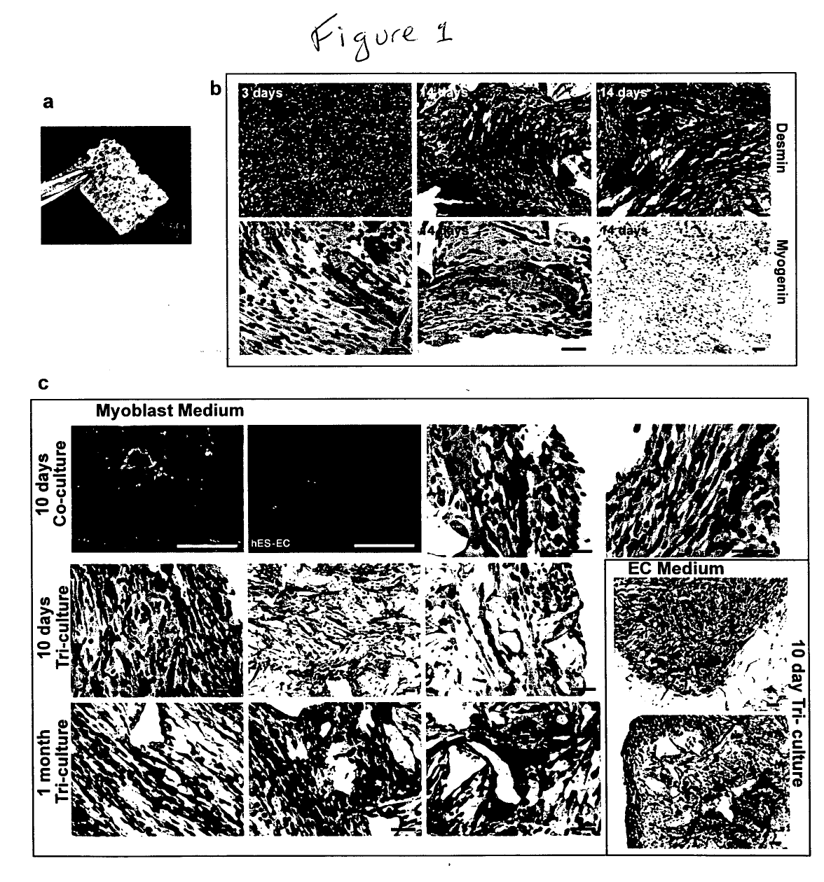

In one embodiment, the invention is a tissue engineered construct including muscle cells, for example, skeletal muscle cells, smooth muscle cells, myoblasts or cardiomyocytes, and endothelial cells, e.g., embryonic endothelial cells (embryonic stem cell-derived endothelial cells), umbilical vein endothelial cells, or endothelial cells isolated from more developed tissue, for example, aortic endothelial cells, seeded on a three dimensional support matrix. For example, a 3D co-culture system may be employed in which the cells are mixed and seeded on highly porous, 3D biodegradable polymer scaffolds. The sponge-like scaffolds may include 50% poly-(L-lactic acid) (PLLA) and 50% polylactic-glycolic acid (PLGA), with pore sizes of, for example, 225-500 μm (Levenberg, et al., Proc Natl Acad Sci USA (2003) 100, 12741-12746, the contents of which are incorporated herein by reference) (FIG. 1A).

In one embodiment, the muscle cells and endothelial cells are mammalian cells. For example, the muscle cells may be human, bovine, or mouse cells. The endothelial cells may be human embryonic stem cell (hESC)-derived endothelial cells (Levenberg, et al., Proc Natl Acad Sci USA (2002) 99, 4391-4396, the contents of which are incorporated by reference herein), and the umbilical vein endothelial cells may be human umbilical vein endothelial cells (HUVEC). Cells of any type useful with the techniques of the invention may be derived from any mammal, for example, humans, mice, rats, pigs, dogs, cats, cows, monkeys, chimpanzees, and other mammals that are commonly domesticated or used in laboratory research. For example, endothelial cells may be retrieved from bovine aortic tissue.

In one embodiment, human embryonic endothelial cells are produced by culturing human embryonic stem cells in the absence of LIF and bFGF to stimulate formation of embryonic bodies, and isolating PECAM1 positive cells from the population. HUVEC, myoblasts, cardiomyocytes, smooth muscle cells, and fibroblasts may be isolated from tissue according to methods known to those skilled in the art or purchased from cell culture laboratories such as Cambrex Biosciences or Cell Essentials.

The cells may be seeded directly onto a polymer matrix, for example, a sponge, which is then implanted into the desired tissue site. Alternatively, the cells may be mixed with a gel which is then absorbed onto the interior and exterior surfaces of the matrix and which may fill some of the pores of a spongy or other porous matrix. Capillary forces will retain the gel on the matrix before hardening, or the gel may be allowed to harden on the matrix to become more self-supporting. Alternatively, the cells may be combined with a cell support substrate in the form of a gel and optionally including extracellular matrix components. An exemplary gel is Matrigel™, from Becton-Dickinson. Matrigel™ is a solubilized basement membrane matrix extracted from the EHS mouse tumor (Kleinman, H. K., et al., Biochem. 25:312, 1986). The primary components of the matrix are laminin, collagen I, entactin, and heparan sulfate proteoglycan (perlecan) (Vukicevic, S., et al., Exp. Cell Res. 202:1, 1992). Matrigel™ also contains growth factors, matrix metalloproteinases (MMPs [collagenases]), and other proteinases (plasminogen activators [PAs]) (Mackay, A. R., et al., BioTechniques 15:1048, 1993). The matrix also includes several undefined compounds (Kleinman, H. K., et al., Biochem. 25:312, 1986; McGuire, P. G. and Seeds, N. W., J. Cell. Biochem. 40:215, 1989), but it does not contain any detectable levels of tissue inhibitors of metalloproteinases (TIMPs) (Mackay, A. R., et al., BioTechniques 15:1048, 1993). Alternatively, the gel may be growth-factor reduced Matrigel, produced by removing most of the growth factors from the gel (see Taub, et al., Proc. Natl. Acad. Sci. USA (1990); 87 (10:4002-6). In another embodiment, the gel may be a collagen I gel, alginate, or agar. Such a gel may also include other extracellular matrix components, such as glycosaminoglycans, fibrin, fibronectin, proteoglycans, and glycoproteins. The gel may also include basement membrane components such as collagen IV and laminin. Enzymes such as proteinases and collagenases may be added to the gel, as may cell response modifiers such as growth factors and chemotactic agents.

As disclosed above, the cells are seeded on porous scaffolds, for example, a mixture of PLLA and PLGA. Other biocompatible polymers may also be employed for use with the invention. Suitable biodegradable matrices are well known in the art and include collagen-GAG, collagen, fibrin, PLA, PGA, and PLA-PGA co-polymers in any ratio. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides. Non-biodegradable polymers may also be used as well. Other non-biodegradable, yet biocompatible polymers include polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, and poly(ethylene oxide). Co-polymers, mixtures, and adducts of any of the above may also be employed. Those skilled in the art will recognize that this is an exemplary, not a comprehensive, list of polymers appropriate for tissue engineering applications.

PLA, PGA and PLA/PGA copolymers are particularly useful for forming the biodegradable matrices. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. PGA is the homopolymer of glycolic acid (hydroxyacetic acid). In the conversion of glycolic acid to poly(glycolic acid), glycolic acid is initially reacted with itself to form the cyclic ester glycolide, which in the presence of heat and a catalyst is converted to a high molecular weight linear-chain polymer. The erosion of the polyester matrix is related to the molecular weights. The higher molecular weights, weight average molecular weights of 90,000 or higher, result in polymer matrices which retain their structural integrity for longer periods of time; while lower molecular weights, weight average molecular weights of 30,000 or less, result in both slower release and shorter matrix lives. For example, poly(lactide-co-glycolide) (50:50) degrades in about six weeks following implantation.

Co-polymers, mixtures, and adducts of any the above polymers may also be used in the practice of the invention. Indeed, co-polymers may be particularly useful for optimizing the mechanical and chemical properties of the matrix. For example, a polymer with a high affinity for stem cells may be combined with a stiffer polymer to produce a matrix having a desired stiffness. Mixtures of PLA and PLGA are disclosed above; in another embodiment, PLA may be combined with poly(caprolactone). Both the choice of polymer and the ratio of polymers in a co-polymer may be adjusted to optimize the stiffness of the matrix.

In some embodiments, the matrix may be formed with a microstructure similar to that of the extracellular matrix that is being replaced. The molecular weight, tacticity, and cross-link density of the matrix may also be regulated to control both the mechanical properties of the matrix and the degradation rate (for degradable scaffolds). The mechanical properties may also be optimized to mimic those of the tissue at the implant site. The shape and size of the final implant should be adapted for the implant site and tissue type. The matrix may serve simply as a delivery vehicle for the stem cells or may provide a structural or mechanical function. The matrix may be formed in any shape, for example, as particles, a sponge, a tube, a sphere, a strand, a coiled strand, a capillary network, a film, a fiber, a mesh, or a sheet.

The porosity of the matrix may be controlled by a variety of techniques known to those skilled in the art. The minimum pore size and degree of porosity is dictated by the need to provide enough room for the cells and for nutrients to filter through the matrix to the cells. The maximum pore size and porosity is limited by the ability of the matrix to maintain its mechanical stability after seeding. As the porosity is increased, use of polymers having a higher modulus, addition of stiffer polymers as a co-polymer or mixture, or an increase in the cross-link density of the polymer may all be used to increase the stability of the matrix with respect to cellular contraction.

The matrices may be made by any of a variety of techniques known to those skilled in the art. Salt-leaching, porogens, solid-liquid phase separation (sometimes termed freeze-drying), and phase inversion fabrication may all be used to produce porous matrices. Fiber pulling and weaving (see, e.g. Vacanti, et al., (1988) Journal of Pediatric Surgery, 23: 3-9) may be used to produce matrices having more aligned polymer threads. Those skilled in the art will recognize that standard polymer processing techniques may be exploited to create polymer matrices having a variety of porosities and microstructures.

In an exemplary embodiment, a cell response modifier such as a growth factor or a chemotactic agent may be added to the polymer matrix. Such a modifier, for example, vascular endothelial-derived growth factor, may be used to promote differentiation of the embryonic endothelial cells. Alternatively, the modifier may be selected to recruit cells to the matrix or to promote or inhibit specific metabolic activities of cells recruited to the matrix. Exemplary growth factors include activin-A, retinoic acid, epidermal growth factor, bone morphogenetic protein, TGFβ, hepatocyte growth factor, platelet-derived growth factor, TGFα, IGF-I and II, hematopoetic growth factors, heparin binding growth factor, peptide growth factors, erythropoietin, interleukins, tumor necrosis factors, interferons, colony stimulating factors, and basic and acidic fibroblast growth factors. In some embodiments it may be growth factors such as nerve growth factor (NGF) or muscle morphogenic factor (MMP). The particular growth factor employed should be appropriate to the desired cell activity. The regulatory effects of a large family of growth factors are well known to those skilled in the art.

Alternatively or in addition, the construct may include one or more bioactive agents, biomolecules, or small molecules. For example, the construct may include an agent that promotes cell adhesion. Exemplary agents are well known to those skilled in the art and include fibronectin, integrins, and oligonucleotides, such as those containing RGD, that promote cell adhesion.

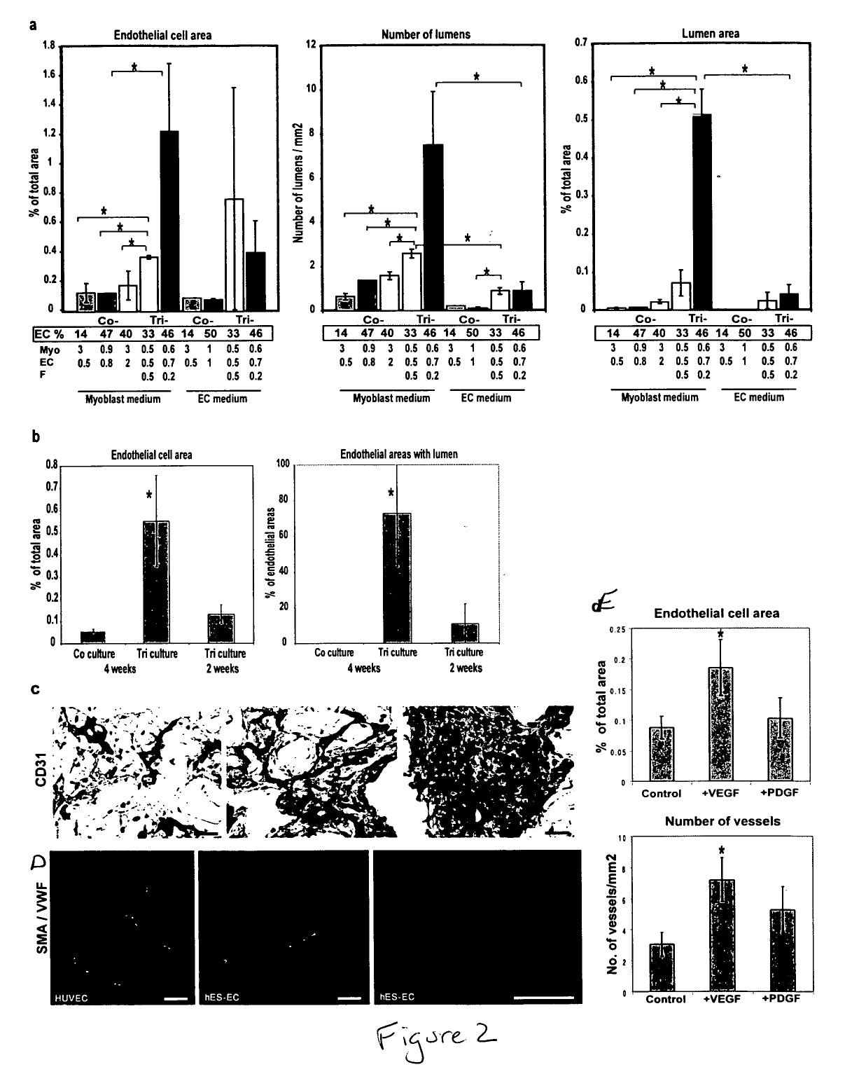

As indicated by desmin immunostaining of cross sections of constructs 3 days after seeding with myoblasts and HUVEC, mouse myoblasts were able to attach and grow onto these scaffolds (FIG. 1B). By 14 days, the myoblasts had differentiated and formed partially aligned, elongated and multinucleated myotubes. Some of the myotubes further differentiated and became myogenin-positive (36%±3% of total nuclei) (FIG. 1B). When endothelial cells (either hESC-derived or HUVEC) were cultured onto the scaffolds with the myoblasts, the endothelial cells (CD31 positive) organized into tubular structures in between the myoblasts and throughout the construct, forming vessel networks within the engineered muscle tissue in vitro (FIG. 1C). Myoblast medium promoted both differentiation of the muscle cells (27%±6% myogenin positive nuclei) and formation of endothelial lumens in the constructs (FIG. 2A), while endothelial medium alone did not support differentiation of the muscle cells (2%±1% myogenin positive nuclei) and inhibited endothelial lumen formation (FIG. 2A).

Association of pericytes or smooth muscle cells with the vessels stabilizes vessels (Darland, et al., Angiogenesis (2001) 4, 11-20; Carmeliet, Nat Med (2003) 9, 653-660; Jain, Nat Med (2003) 9, 685-693; Sieminski, et al., Tissue Eng (2002) 8, 1057-1069; Koike, et al., Nature (2004) 428, 138-139; Black, et al., FASEB J (1998) 12, 1331-1340; Shinoka, et al., Artif Organs (2002) 26, 402-406), and endothelial cells can induce the differentiation of undifferentiated mesenchymal cells into smooth muscle cells (Flamme, et al., J Cell Physiol (1997) 173, 206-210; Rossant, et al., Curr Opin Genet Dev (2003) 13, 408-412; Hirschi, et al., J Cell Biol (1998) 141, 805-814; Darland, et al., Dev Biol (2003) 264, 275-288). We have found that embryonic fibroblasts can promote the formation of vessels characterized by lumen structures in engineered muscle tissue. As shown in FIG. 2A, addition of embryonic fibroblasts to the cultures, together with myoblasts and endothelial cells, significantly promoted vascularization of the engineered muscle, as indicated by increases in total area of endothelial cells and number and size of endothelial lumens, compared to co-cultured constructs seeded with myoblasts and endothelial cells only. The effect of the fibroblasts was dependent on the cell ratios and medium conditions (FIG. 2A, “tri-cultures”). The inductive effect of embryonic fibroblasts on endothelial vessels is shown also in tri-culture samples that were seeded with a lower ratio of endothelial cells (of the total cell number) compared to cultures of myoblasts and endothelial cells only (FIG. 2A).

To analyze the stability of the in vitro vessel-like structures formed in the muscle constructs, we examined tri-culture constructs (a combination of myoblasts, fibroblasts and endothelial cells) at two weeks and one month. Large vessel structures (>1500 μm²) were evident only in the one-month-old tri-culture constructs (FIG. 1 C). In addition, tri-cultures grown for one month had a two-fold increase in number of endothelial structures, a greater surface area covered by endothelial cells and a higher percentage of vessel-like structures with lumens, compared to two week tri-cultures (FIG. 2B).

These results suggest that addition of embryonic fibroblasts promoted stabilization of the vessel structures over time. Double labeling for vWF and smooth muscle actin (SMA) on cross-sections of constructs seeded with endothelial cells and embryonic fibroblasts showed that fibroblasts in the cultures had indeed differentiated into smooth muscle cells and were co-localized around endothelial cells in vessel-like structures (FIG. 2C-2D). Quantitative analysis of the double staining revealed that 65.7±8.8% of endothelial vessel-like structures in the constructs had associated smooth muscle cells.

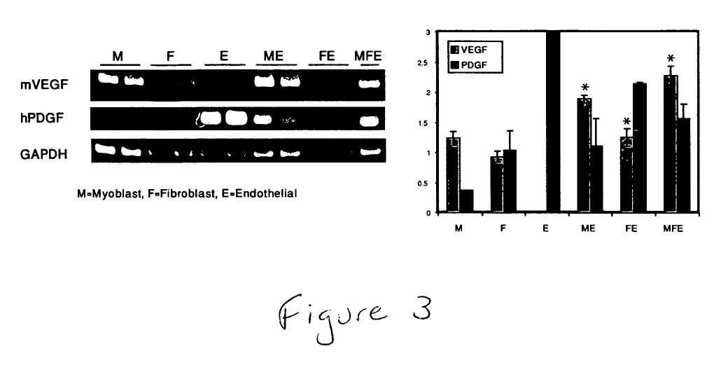

To study the expression of key vasculogenic and angiogenic factors in the 3D-muscle constructs we analyzed the expression of VEGF and PDGF-B at the mRNA level in muscle constructs after two weeks in culture. The RT-PCR results indicated that addition of human endothelial cells to myoblast or fibroblast cultures resulted in an increase in mouse VEGF expression in the construct. Moreover, tri-cultures that included embryonic fibroblasts had higher levels of VEGF mRNA than myoblast-endothelial co-cultures (FIG. 3). The increased VEGF expression is consistent with the increase in the endothelial network observed in the tri-cultures, and could be one of the factors affecting the induction of vascularization of the constructs. Indeed, addition of VEGF into the medium resulted in a larger area covered by endothelial cells and an increase in the number of vessel-like endothelial tubular structures in the constructs (FIG. 2E). Depletion of VEGF from the medium resulted in a decreased number of vessel structures, while addition of fibroblasts to cultures without VEGF supplementation restored vessel formation (data not shown).

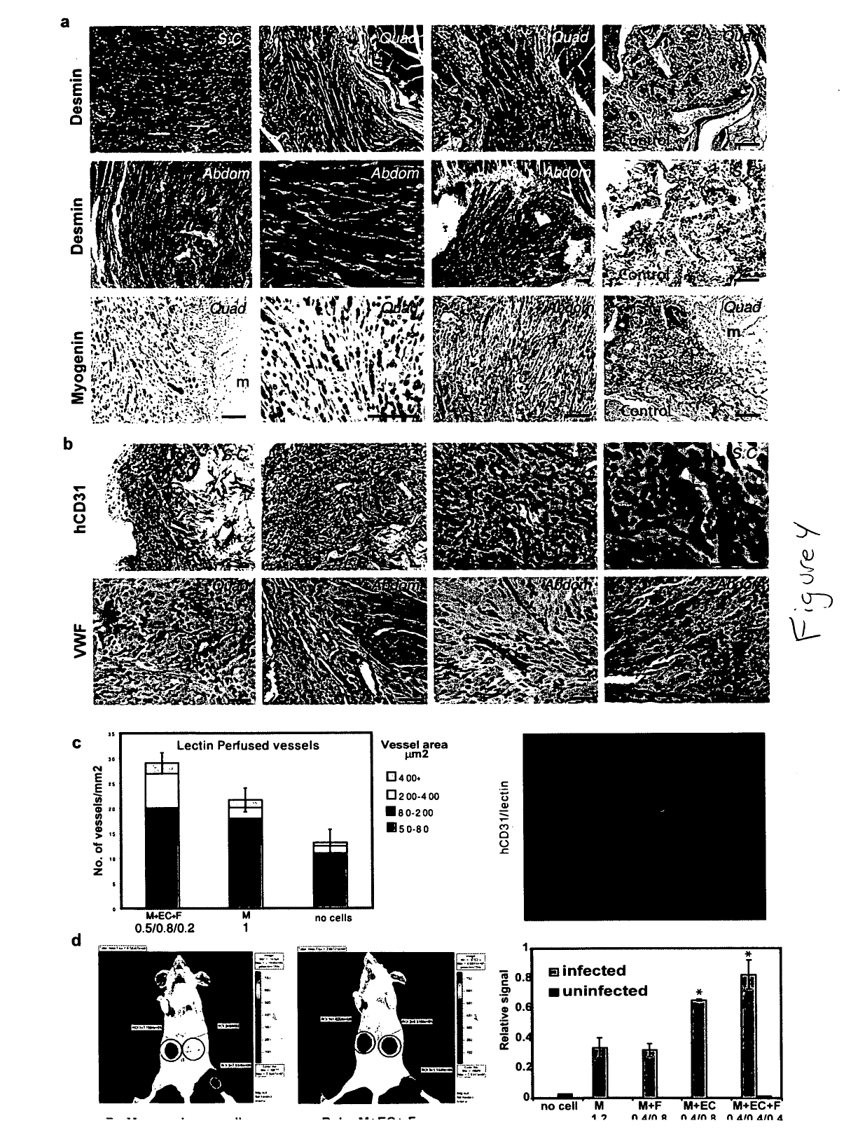

To assess the therapeutic potential of our approach, we used three models to analyze survival, differentiation, integration and vascularization of the implant in vivo: (i) Subcutaneous implantation in the back of SCID mice, (ii) Intramuscular implantation into the quadriceps muscle of nude rats, and (iii) Replacement of anterior abdominal muscle segment of nude mice with the construct. In all three models, the muscle implant continued to differentiate in vivo. The implanted myotubes were elongated, aligned, and multi-nucleated (6-8 nuclei), with a high percentage of myogenin-positive myotubes (67%±9%)(FIG. 4A). Control implants, containing no cells or fibroblasts, contained no desmin-positive myotubes or myogenin-positive nuclei within the scaffold area (FIG. 4A). To further ensure that the myotubes observed within the scaffold were derived from implanted cells rather than invading host cells, BrdU was incorporated in the tissue engineered constructs prior to implantation. BrdU labeling confirmed that the implanted myoblasts had indeed survived and differentiated to populate the scaffold (data not shown). In most cases, particularly in the abdominal muscle, the implanted myotubes were in close contact with the host muscle, with very thin and sometimes barely detectable fibrous tissue around the implant (FIG. 4A). The myotubes in the implanted area were relatively long and thick and in many cases appeared to have re-oriented to align with the fibers of the host tissue (FIG. 4A).

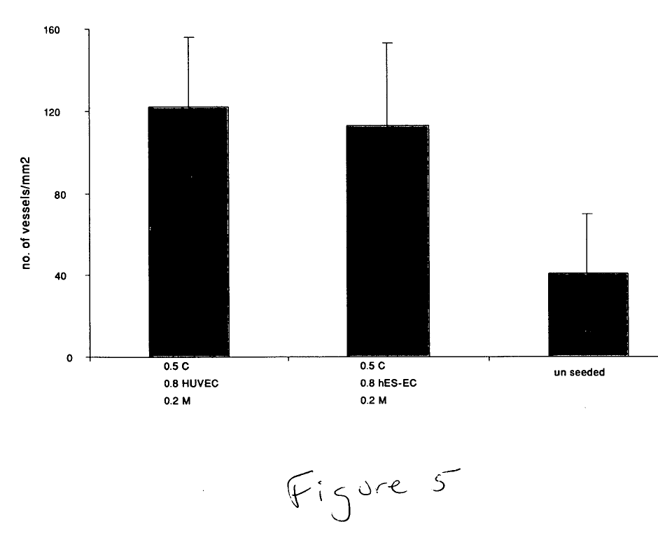

The constructs were permeated with host blood vessels (FIG. 4B). Quantification of the number of endothelial vessel-like structures in intramuscularly implants two weeks after implantation indicated that there was no significant difference between constructs seeded with HUVEC or hESC-derived endothelial cells (FIG. 5). Staining of subcutaneous implants with anti-human specific endothelial antibodies (anti-CD31) demonstrated the presence of vessels (between elongated myotubes), which were lined by implanted human endothelial cells. Moreover, construct-derived vessels contained intraluminal red blood cells, suggesting that vessels had anastomosed with the host vasculature (FIG. 4B). To analyze functional vessels, labeled lectin was injected two weeks after implantation and perfused vessels were counted. The results indicated that 41%±12% of human CD31 positive vessels (implant-derived vessels) were functional and lectin-perfused. Quantification of the total number of perfused vessels (host and implant derived) indicated that constructs seeded with endothelial cells had 30±2 functional vessels per square millimeter compared to 21±2 vessels in constructs seeded with muscle cells only (n=3, p<0.01). Size distribution of functional vessels in the implant showed that including endothelial cells in the scaffolds also increased the number of larger or stabilized vessels in the muscle implants (FIG. 4C, 4D). These results demonstrate that pre-endothelialization of the construct promotes implant vascularization and thus can affect blood perfusion to the muscle implant as well as implant survival in vivo.

To further evaluate tissue-engineered muscle construct survival and integration in vivo, we employed a luciferase-based imaging system. The in vivo imaging system (IVIS) works by detecting light generated by the interaction of systemically administered luciferin with locally produced luciferase. Constructs were infected with Adeno-Associated Virus (AAV) vector encoding luciferase for 48 hours prior to transplantation. Detection of luciferase expression in the constructs indicated no difference between the various muscle-constructs in vitro. The constructs were then placed in situ in the anterior abdominal muscle walls of nude mice. Three weeks following surgery, the mice received luciferin to assess perfusion to the tissue-engineered construct. There was a minimal signal detected in areas that did not receive gene transfer or in control constructs that were not seeded with cells. However strong signals were detected from areas either directly transduced with AAV-luciferase (as controls) or transplanted with virally transduced cells indicating perfused vessels. By using the luciferase system, we were able to non-invasively determine the degree to which different constructs continued to survive (and express luciferase) and maintain vascular connections with the recipient in order to receive systemically delivered luciferin. The results described in FIGS. 4E and F show that the relative signal detected in implants seeded with endothelial cells was higher than in myoblast-only implants. Coupled with similar pre-implantation levels of luciferase expression and with the histological evidence of increased functional vascularity, the results suggest that the increased signal in pre-endothelialized samples is related to increased perfusion and survival of the tissue engineered muscle constructs. Analysis of cell survival in the implant using TUNEL staining indicated a two fold increase in the number of apoptotic cells in muscle-only implants compared to pre-endothelialized implant (36±9 and 19±7 cells per implant (n=3))

The approach that we have developed enables formation and stabilization of endothelial vessel networks in 3D engineered skeletal muscle tissue in vitro. The overall in vivo results show that pre-vascularization of the implants improves implant vascularization and survival. Unlike previous studies demonstrating endothelial differentiation within fibroblast culture and fibroblast differentiation into pericytes (Darland, 2001; Carmeliet, 2003; Jain, 2003; Sieminski, et al., 2002; Koike, 2004), this study demonstrates engineering of 3D vascularized skeletal muscle construct with formation of the endothelial networks throughout and in between differentiating skeletal muscle fibers. This study emphasizes the importance of multi-cell cultures in providing appropriate signals for vascular organization in skeletal muscle tissue. Moreover, it provides important evidence for the potential of endothelial co-cultures in promoting in vivo vascularization of engineered tissues. In addition to tissue vacularization, co-cultures with endothelial cells may be important for inducing differentiation of engineered tissues, as embryonic endothelial cells are critical for the earliest stages of organogenesis (Lammert, et al., Science (2001) 294, 564-567; Matsumoto, et al., Science (2001) 294, 559-563). We believe that this approach could have potential applications in tissue engineering and may provide an important tool for studying multi-cellular processes such as tissue vascularization in vitro.

### EXAMPLES

Cell Culture

Mouse myoblast cells (C2C12) (Yaffe, et al., Nature (1977) 270, 725-727; Blau, et al., Science (1985) 230, 758-766, the contents of both of which are incorporated herein by reference) were cultured in DMEM supplemented with 10% fetal bovine serum (FBS), 10% calf serum, and 2.5% HEPES buffer (myoblast medium). Human umbilical vein endothelial cells (HUVEC) were cultured in endothelial cell medium (EGM-2; Cambrex Biosciences). Mouse embryonic fibroblasts (MEF) (Cell Essentials, Boston) were cultured in DMEM supplemented with 10% FBS (embryonic fibroblast medium). HESC-derived CD31+endothelial cells were isolated as described (Levenberg, et al., 2002) and cultured in endothelial cell medium.

Polymer Scaffolds

Porous sponges composed of 50% poly-(L-lactic acid) (PLLA) and 50% polylactic-glycolic acid (PLGA) were fabricated as described (Levenberg, et al., 2003) with pore sizes of 225-500 μm and 93% porosity. The PLGA was selected to degrade quickly (˜3 weeks) to facilitate cellular ingrowth, whereas the PLLA was chosen to provide mechanical support to 3D structures. The degradation time of the composed sponges is ˜6 months. Biocompatibility of PLLA and PLGA porous scaffolds was previously shown (Holder, et al., J Biomed Mater Res (1998) 41, 412-421). For seeding, the desired number of cells were pooled and resuspended in 7-15 μl of a 1:1 mixture of culture medium and growth factor-reduced Matrigel (BD Biosciences). This suspension was allowed to absorb into the sponges, after which the sponges were incubated for 30 minutes at 37° C. to allow solidification of the gel. Culture medium was then added, the sponges were detached from the bottom, and incubated at 37° C. on a XYZ shaker. The medium was changed every other day. At the conclusion of the experiments, samples were fixed in 10% formalin and subsequently embedded in paraffin for sectioning or were transplanted into mice or rats.

Implantation of Muscle Constructs

Male 5- to 6-week old SCID mice (CB.17 SCID) were anesthetized with 2.5% isoflurane in balanced oxygen, after which a construct was implanted subcutaneously on each side lateral to the dorsal midline region of each mouse. For intramuscular implantation, constructs were implanted into the outer quadriceps muscle of the right hand side of Male 5-7 week old nude rats. Sutures of 6-0 Prolene in a simple interrupted pattern were used to prevent movement of the constructs from the muscle site, and the skin was closed using surgical staples. Two to eight weeks after implantation, the mice or rats were euthanized and the implants were retrieved. Samples were fixed in 10% neutral buffered-formalin, processed routinely and sectioned at 4 μm prior to staining. For perfusion analysis: 1. Lectin perfusion: Lectin HPA (Helix pomatia agglutinin) conjugated to Alexa Fluor 594 (Molecular Probes) (0.5 mg /0.25 ml PBS) was injected into the tail vein of anesthesized animals (20 mg/kg body weight). Circulation was allowed for 2 minutes after which the animals were euthanized and the implants were retrieved. Samples were snap frozen (liquid nitrogen) in Cryomatrix (Thermo Shandon) and sections of 6 μm were cut with a cryotome. 2. Luciferase assay (abdominal wall model): Tissue engineered constructs were placed in 12-well culture dishes in 2 ml of medium and 1.0×10⁹ dot blot/cc of AAV-Luciferase added. After 48 hours, the constructs were washed with two volumes of PBS. As a control, Luciferin (150 μl of 5 mg/ml) was added to each well. After incubation for 11 minutes, the constructs were imaged in the Xenogen IVIS device at a three-minute exposure. Luminescence was determined by calculating the flux (photons/sec/cm²) overlying each construct. Immediately thereafter, the constructs were implanted into isoflurane-anasthetized mice by creation of a 3 mm×3 mm defect in the anterior abdominal wall of the mice in line with the inferior epigastric artery. The construct was then sutured in place using four 7-0 prolene sutures attached to each corner of the construct. The ventral skin was sutured and the animal recovered. At various intervals following surgery, the animals were imaged using the Xenogen IVIS device. Mice were anesthetized using an IM injection of ketamine and xylazine. Luciferin (150 μl of 5 mg/ml) was injected subcutaneously on the dorsal surface of the mouse and allowed to circulate for 11 minutes before the animals were imaged. Luminescence was calculated by determining the photon flux. A 1.0 cm² area was chosen arbitrarily as the standard. The ratio of the flux from the tissue-engineered construct relative to the hind limb was calculated. After the final imaging session, the animals were euthanized and the implants were retrieved and placed in 10% formalin prior to routine processing and histological sectioning. Unseeded scaffolds were used as controls and showed host-cell infiltration (including fibroblasts and blood vessels) (see Holder, et al., 1998).

Tissue Processing and Immunohistochemical Staining

Tissue constructs were fixed for 6 hours in 10% neutral buffered formalin, routinely processed, and embedded in paraffin. Transverse sections (4 -μm) were placed on silanized slides for immunohistochemistry or staining with hematoxylin and eosin. Immunohistochemical staining was carried out using the Biocare Medical Universal HRP-DAB kit (Biocare Medical, Walnut Creek, Calif.) according to the manufacturer's instructions, with prior heat-treatment at 95° C. for 20 minutes in ReVeal buffer (Biocare Medical) for epitope recovery. For immunofluorescent staining, the secondary antibodies used were Alexa Fluor (Molecular Probes) and Cy-3 (Jackson Laboratories) followed by DAPI (Sigma) nuclear staining. The primary antibodies were anti-human: CD31 (1:20); desmin (1:150); α-smooth muscle actin (1:50); vWF (1:200) (all from Dako, Carpinteria, Calif.); or VWF (Chemicon) (1:100). Staining with α-smooth muscle actin antibody (as well as other SMC markers) (to identify fibroblasts differentiation into SMC), could not be done in the presence of C₂C₁₂ cells due to expression of these markers by the C₂C₁₂ myoblasts. The vWF antibody was used following deparaffinization+trypsin treatment for epitope recovery. For labeling implanted myoblasts, myoblast culture medium was supplemented with 10 μm of 5′-bromo-2′-deoxyuridine (BrdU) (Sigma) and applied to 60% confluent dish cultures for 16 hours. Cells were washed and seeded on scaffolds as described. Tissue sections were stained using mouse anti-BrdU antibodies (1:1000) as described, but with treatment with 2N HCl and 0.5% Triton X-100 for 30 minutes at 37° C. to denature the DNA, prior to addition of the antibodies. DeadEnd colorimetric TUNEL system (Promega) was used to stain apoptotic cells.

Reverse Transcription (RT)-PCR analysis

Total RNA was isolated by an RNEasy Mini Kit (Qiagen, Chatsworth, Calif.), using the “isolation from tissue” protocol. RT-PCR was carried out using a Qiagen OneStep RT-PCR kit with 10 units RNase inhibitor (Gibco) and 40 ng RNA. To ensure semi quantitative results of the RT-PCR assays the number of PCR cycles for each set of primers was checked to be in the linear range of the amplification. Primer sequences: Mouse VEGF: 5′ CCT CCG AAA CCA TGA ACT TTC TGC TC, and 5′ CAG CCT GGC TCA CCG CCT TGG CTT. Human PDGF: 5′ GGA GCA TTT GGA GTG CGC CT, and 5′0 ACA TCC GTG TCC TGT TCC CGA.

The amplified products were separated on 1.2% agarose gels with ethidium bromide (E-Gel, Invitrogen, Gaithersburg, Mass.). Mean pixel intensities of each band were measured and normalized to mean pixel intensities of GAPDH band. The values for two experiments (performed in duplicate) were then averaged and graphed with standard deviation. P values were calculated using student t-test.

Image Analysis

Overlapping microscopic pictures were taken at a magnification of 100× so that the entire area of the sample was covered. An imaging software (AxioVision 3.1, Carl Zeiss) was used to determine the area of endothelial cells, the area of vessels or lumen, and the total sample area. The number of structures with lumen was counted manually. For co-localization analysis, 3-6 randomly chosen 20× fields were analyzed using OpenLab (Density Slice Module) image analysis software (ImproVision) to quantify endothelial cell positive areas with and without co-localization of smooth-muscle cell positive areas. An endothelial cell positive area was identified by the presence of a vWF-positive vessel-like structure with a lumen.

Tissue Engineering of Cardiac Tissue

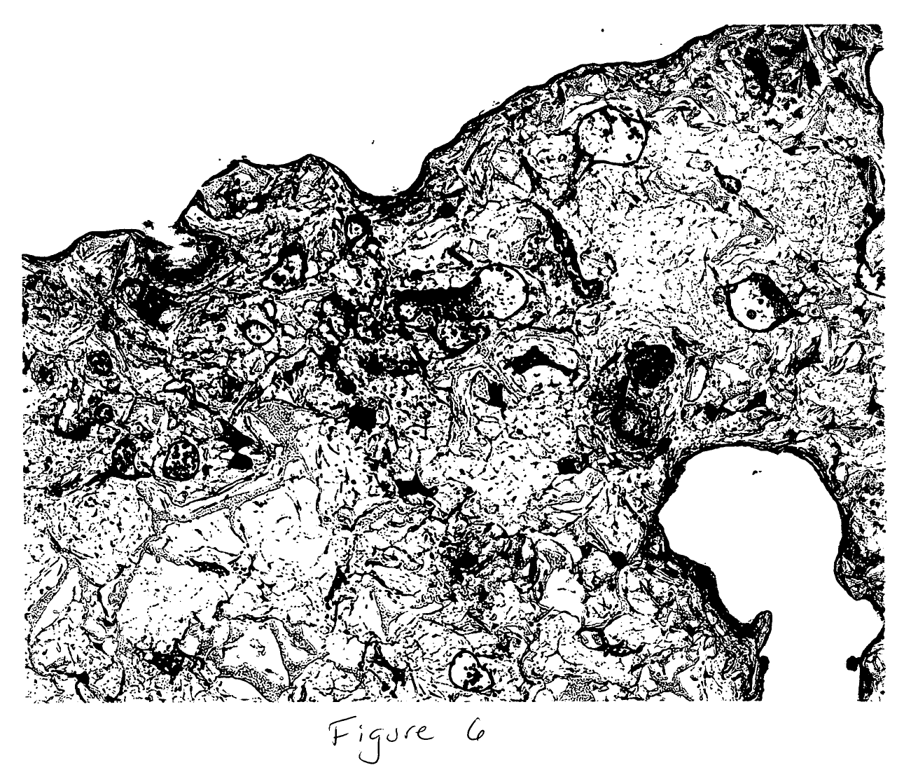

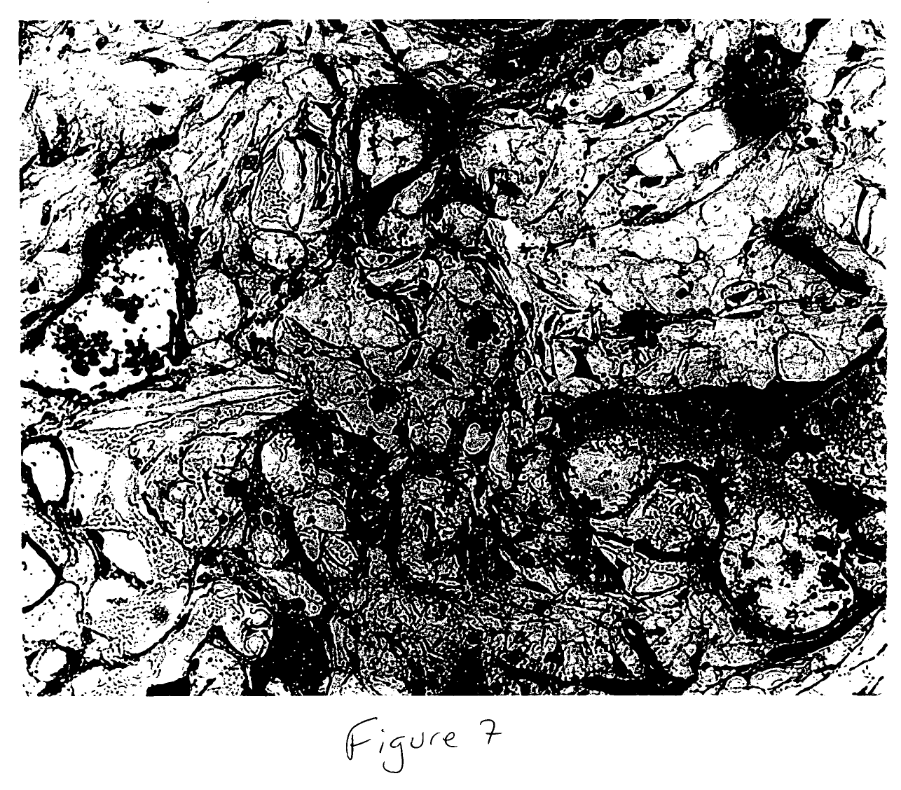

Scaffolds were seeded with 0.2 million cardiomyocytes and 0.4 million HUVEC. After fourteen days incubation in 50% endothelial medium/50% cardiac medium (DMEM/10% fetal calf serum/1% HEPES buffer), the scaffolds were sectioned and stained with anti-CD31 antibodies (FIGS. 6 and 7). The figures show the proliferation of endothelial cells and the development of vessel like structures with a lumen.

Tissue Engineering with Adult Endothelial Cells

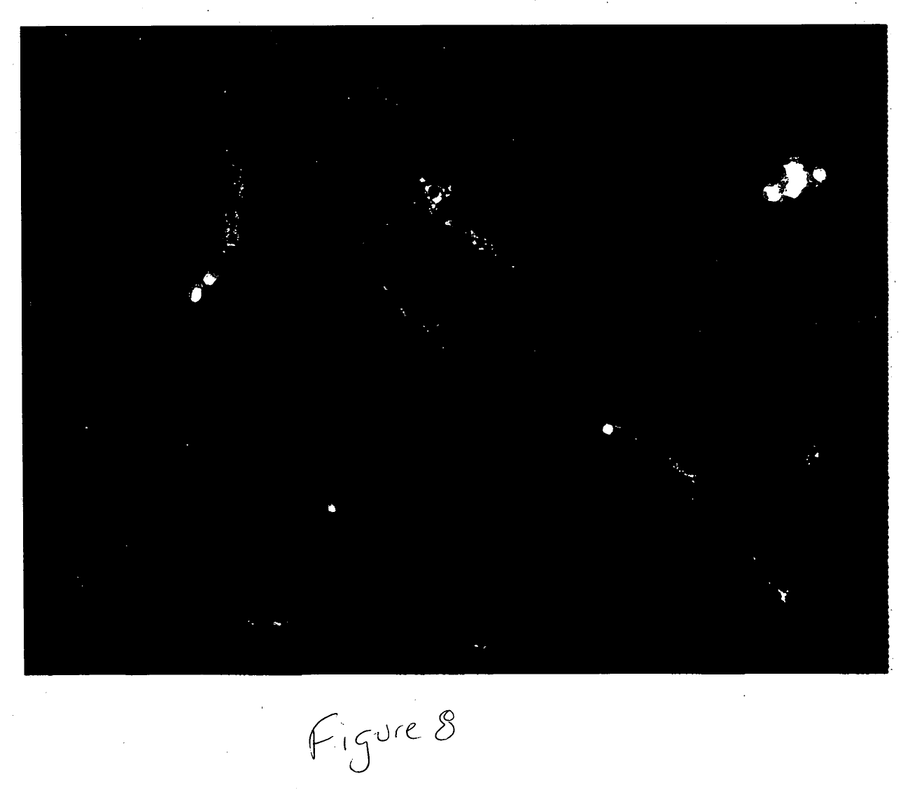

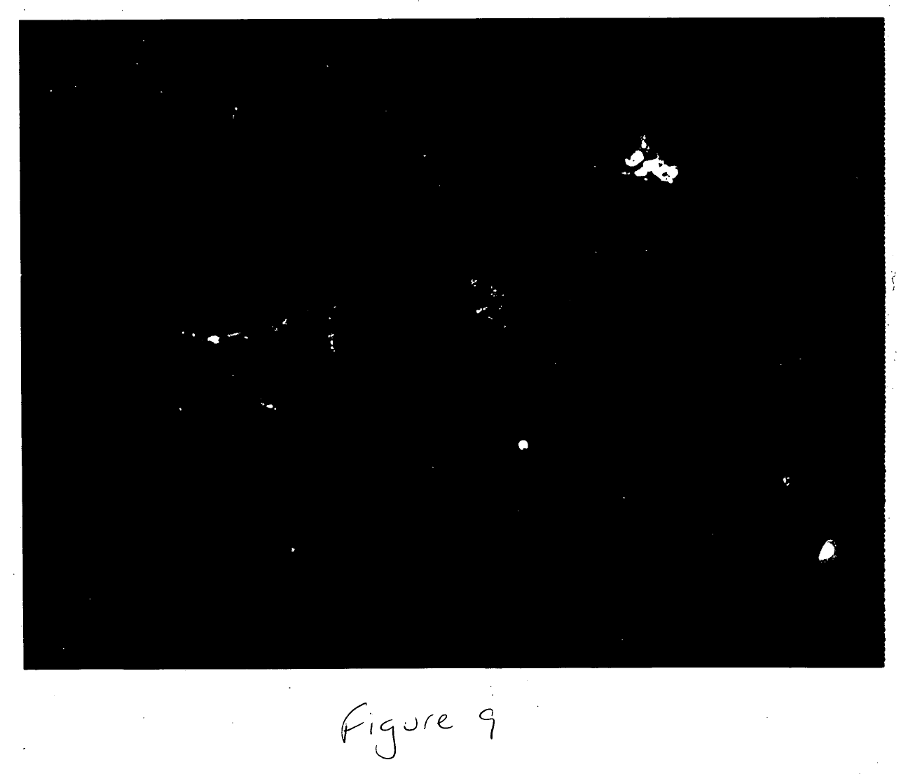

Scaffolds were seeded with bovine aortic endothelial cells and mouse embryonic fibroblasts (0.5×10⁶ each). After fourteen days incubation in 50% endothelial medium/50% EF medium (DMEM/10% fetal calf serum), the scaffolds were sectioned and stained to reveal α-smooth muscle actin and vWF (FIGS. 8 and 9) as described above. The figures demonstrate the differentiation of fibroblasts into smooth muscle cells and their co-localization about endothelial cells in vessel-like structures.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A tissue engineered construct, comprising:
endothelial cells;
muscle cells; and
a three-dimensional support matrix on which the endothelial cells and the muscle are seeded.

2. The construct of claim 1, wherein the construct promotes the formation of one or more of smooth, skeletal, and cardiac muscle tissue.

3. The construct of claim 1, wherein the endothelial cells are embryonic stem cell-derived endothelial cells or umbilical vein endothelial cells;

4. The construct of claim 2, wherein the embryonic stem cell-derived endothelial cells are mammalian embryonic stem cell-derived endothelial cells.

5. The construct of claim 2, wherein the embryonic stem cell-derived endothelial cells are human embryonic stem cell-derived endothelial cells.

6. The construct of claim 2, wherein the umbilical vein endothelial cells are human or mouse umbilical vein endothelial cells.

7. The construct of claim 1, wherein the endothelial cells are mammalian aortic endothelial cells.

8. The construct of claim 1, wherein the muscle cells are mammalian myoblasts.

9. The construct of claim 1, wherein the muscle cells are human or mouse myoblasts.

10. The construct of claim 1, wherein the muscle cells are cardiomyocytes.

11. The construct of claim 1, wherein the muscle cells are skeletal muscle cells.

12. The construct of claim 1, wherein the muscle cells are smooth muscle cells.

13. The construct of claim 1, further comprising fibroblasts.

14. The construct of claim 13, wherein the fibroblasts are embryonic fibroblasts.

15. The construct of claim 13, wherein the fibroblasts are human or mouse embryonic fibroblasts.

16. The construct of claim 1, wherein the three-dimensional support matrix comprises a mixture of poly(L-lactic acid) and poly(lactic acid-co-glycolic acid).

17. The construct of claim 1, wherein the three-dimensional support matrix comprises a 50:50 mixture of poly(L-lactic acid) and poly(lactic acid-co-glycolic acid).

18. The construct of claim 1, wherein the three-dimensional support matrix is biodegradable or non-biodegradable.

19. The construct of claim 18, wherein the three-dimensional support matrix comprises collagen-GAG, collagen, fibrin, PLA, PGA, PLA-PGA co-polymers, poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of the above, mixtures of the above, and adducts of the above.

20. The construct of claim 1, wherein the three-dimensional support matrix further comprises a coating including an agent that promotes cell adhesion.

21. The construct of claim 20, wherein the agent that promotes cell adhesion is selected from fibronectin, integrins, and oligonucleotides that promote cell adhesion.

22. The construct of claim 1, wherein the cells are combined with growth-factor reduced Matrigel.

23. The construct of claim 1, further comprising a gel that coats internal and external surfaces of the three-dimensional support matrix.

24. The construct of claim 23, wherein the gel is selected from collagen gel, alginate, agar, growth factor-reduced Matrigel, and MATRIGEL™.

25. The construct of claim 23, wherein the gel further comprises one or more of laminin, fibrin, fibronectin, proteoglycans, glycoproteins, glycosaminoglycans, chemotactic agents, or growth factors.

26. The construct of claim 1, further comprising VEGF.

27. The construct of claim 1, further comprising a growth factor.

28. The construct of claim 27, wherein the growth factor is selected from activin-A (ACT), retinoic acid (RA), epidermal growth factor, bone morphogenetic protein, TGF-β, hepatocyte growth factor, platelet-derived growth factor, TGF-β, IGF-I, IGF-II, hematopoietic growth factors, heparin binding growth factor, peptide growth factors, erythropoietin, interleukins, tumor necrosis factors, interferons, colony stimulating factors, acidic and basic fibroblast growth factors, nerve growth factor (NGF), and muscle morphogenic factor.

29. A tissue-engineered muscle construct, comprising:
a three-dimensional support matrix;
a plurality of myotubes disposed within the support matrix; and
at least one endothelial vessel structure disposed within the support matrix.

30. The construct of claim 29, wherein the endothelial vessel structure comprises at least one vessel-like structure having a lumen.

31. A tissue-engineered muscle construct, comprising:
a three-dimensional support matrix;
a plurality of cardiac muscle cells disposed within the support matrix; and
at least one endothelial vessel structure disposed within the support matrix.

32. The construct of claim 31, wherein the endothelial vessel structure comprises at least one vessel-like structure having a lumen.

33. A method of producing a tissue engineered construct, comprising:
providing a population of endothelial cells;
providing a population of muscle cells;
seeding the endothelial cells and the muscle cells on a three-dimensional support matrix; and
culturing the seeded cell support matrix in a predetermined medium for a predetermined period of time.

34. The method of claim 33, wherein seeding comprises suspending the muscle cells and the endothelial cells in growth-factor reduced Matrigel and absorbing a predetermined amount of the suspension into the three-dimensional support matrix.

35. The method of claim 33, wherein the predetermined medium includes one or more of myoblast medium, endothelial cell medium, cardiac cell medium, and embryonic fibroblast medium.

36. The method of claim 35, wherein the myoblast medium comprises DMEM containing 10% fetal bovine serum, 10% calf serum, and 2.5% HEPES buffer.

37. The method of claim 35, wherein cardiac cell medium comprises DMEM containing 10% fetal calf serum and 1% HEPES buffer.

38. The method of claim 35, wherein embryonic fibroblast medium comprises DMEM containing 10% fetal calf serum.

39. The method of claim 35, further comprising supplementing the medium with VEGF.

40. The method of claim 33, wherein the endothelial cells are embryonic stem cell-derived endothelial cells or umbilical vein endothelial cells.

41. The method of claim 40, wherein the embryonic stem cell-derived endothelial cells are mammalian embryonic stem cell-derived endothelial cells.

42. The method of claim 40, wherein the embryonic stem cell-derived endothelial cells are human embryonic stem cell-derived endothelial cells.

43. The method of claim 40, wherein the umbilical vein endothelial cells are human or mouse umbilical vein endothelial cells.

44. The method of claim 33, wherein the endothelial cells are mammalian aortic endothelial cells.

45. The method of claim 33, wherein the muscle cells are mammalian myoblasts.

46. The method of claim 33, wherein the muscle cells are human or mouse myoblasts.

47. The method of claim 33, wherein the muscle cells are cardiomyocytes.

48. The method of claim 33, wherein the muscle cells are smooth muscle cells.

49. The method of claim 33, wherein the muscle cells are skeletal muscle cells.

50. The method of claim 33, wherein the method further comprises providing a population of fibroblasts and wherein seeding comprises seeding the fibroblasts with the endothelial cells and the myoblasts on the three-dimensional support matrix.

51. The method of claim 50, wherein the fibroblasts are mammalian embryonic fibroblasts.

52. The method of claim 50, wherein the fibroblasts are human or mouse embryonic fibroblasts.

53. The method of claim 33, wherein the three-dimensional support matrix comprises a mixture of poly(L-lactic acid) and poly(lactic acid-co-glycolic acid).

54. The method of claim 33, wherein the three-dimensional support matrix comprises a 50:50 mixture of poly(L-lactic acid) and poly(lactic acid-co-glycolic acid).

55. The method of claim 33, wherein the three-dimensional support matrix is biodegradable or non-biodegradable.

56. The method of claim 55, wherein the three-dimensional support matrix comprises collagen-GAG, collagen, fibrin, PLA, PGA, PLA-PGA co-polymers, poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of the above, mixtures of the above, and adducts of the above.

57. The method of claim 33, wherein the three-dimensional support matrix further comprises a coating including an agent that promotes cell adhesion.

58. The method of claim 57, wherein the agent that promotes cell adhesion is selected from fibronectin, integrins, and oligonucleotides that promote cell adhesion.

59. The method of claim 33, wherein seeding comprises suspending the muscle cells and the endothelial cells in a gel and absorbing a predetermined amount of the suspension into the three-dimensional support matrix.

60. The method of claim 59, wherein the gel is selected from collagen gel, alginate, agar, growth factor-reduced Matrigel, and MATRIGEL™.

61. The method of claim 59, wherein the gel further comprises one or more of laminin, fibrin, fibronectin, proteoglycans, glycoproteins, glycosaminoglycans, chemotactic agents, or growth factors.

62. The method of claim 59, further comprising supplementing the medium with a growth factor.

63. The method of claim 62, wherein the growth factor is selected from activin-A (ACT), retinoic acid (RA), epidermal growth factor, bone morphogenetic protein, TGF-β, hepatocyte growth factor, platelet-derived growth factor, TGF-α, IGF-I, IGF-II, hematopoietic growth factors, heparin binding growth factor, peptide growth factors, erythropoietin, interleukins, tumor necrosis factors, interferons, colony stimulating factors, acidic and basic fibroblast growth factors, nerve growth factor (NGF), and muscle morphogenic factor.
